(19) United States
(12) Patent Application Publication
(10) Pub. No.: US 2024/0051918 A1
(43) Pub. Date: Feb. 15, 2024

(54) SEQUENTIAL AND INDEPENDENT SYNTHESIS OF MERCAPTANS AND ASYMMETRICAL SULFIDES IN A SINGLE REACTOR

(71) Applicant: Chevron Phillips Chemical Company LP, The Woodlands, TX (US)

(72) Inventors: Kenneth M. Lassen, Bartlesville, OK (US); Ugochukwu Nwagwu, Houston, TX (US); Daniel M. Hasenberg, Kingwood, TX (US)

(21) Appl. No.: 17/884,774

(22) Filed: Aug. 10, 2022

(51) Int. Cl. C07C 319/20; B01J 23/882; B01J 23/883

(52) U.S. Cl. CPC C07C 319/20; B01J 23/882; B01J 23/883; C07C 2523/882; C07C 2523/883

(57) ABSTRACT

The present disclosure generally relates to processes to produce a mercaptan and an asymmetrical sulfide utilize stacked bed catalyst systems containing CoMo and NiMo, and these processes demonstrate a synergistic reduction in the amount of ethylene, methyl mercaptan, ethyl mercaptan, and H₂S in product mixtures. In aspects, the conversion of limiting reactants in the synthesis of asymmetrical sulfides unexpectedly remain high under increased flow rates through the catalyst bed, and under reduced temperatures and pressures. In further aspects, reactor systems configured for the independent synthesis of mercaptans and asymmetrical sulfides in a single fixed bed catalyst vessel are also disclosed as a simplification of existing reactor systems employing separate reactors for separate mercaptan and sulfide syntheses.

### FIELD OF THE INVENTION

The present disclosure generally relates to processes and reactor systems for producing mercaptans and asymmetrical sulfides utilizing supported catalysts, and related processes and reactor systems utilizing layered CoMo/NiMo catalysts to independently produce a mercaptan and an asymmetrical sulfide.

### BACKGROUND OF THE INVENTION

Transulfidation of symmetrical sulfides utilizing supported cobalt-molybdenum (CoMo) catalysts is a synthetic pathway to asymmetrical sulfides, such as methyl ethyl sulfide. Mercaptan synthesis often can be conducted utilizing cobalt-molybdenum catalysts or nickel-molybdenum (NiMo) catalysts. It would be beneficial to have a single reactor system that is capable of both asymmetrical sulfide synthesis and mercaptan synthesis, and at high yield and selectivity. Accordingly, it is to this end that the present disclosure is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Processes for producing mercaptans and asymmetrical sulfides are disclosed herein. In one aspect, such processes can comprise sequentially (a) flowing a first feed mixture comprising hydrogen sulfide (H₂S) and an olefin through a first catalyst layer of a fixed bed reactor comprising a supported CoMo catalyst, then through a second catalyst layer of the fixed bed reactor comprising a supported NiMo catalyst to produce a first reaction mixture comprising the mercaptan, and (b) flowing a second feed mixture comprising a first symmetrical sulfide and a second symmetrical sulfide through the first catalyst layer and then the second catalyst layer in the fixed bed reactor to produce a second reaction mixture comprising the asymmetrical sulfide.

Reactor systems are also disclosed herein, and in certain aspects can comprise (i) a fixed bed reactor comprising a reactor inlet for a feed stream, a first catalyst layer comprising a supported CoMo catalyst, a second catalyst layer comprising a supported NiMo catalyst, and a reactor outlet for a reaction mixture, (ii) a first feed source comprising a H₂S storage vessel and an olefin storage vessel, and (iii) a second feed source comprising a first symmetrical sulfide storage vessel and a second symmetrical sulfide storage vessel. A flow path for the feed stream begins at the reactor inlet, through the first catalyst layer and then the second catalyst layer, and to the reactor outlet. Generally, a weight ratio of the first catalyst layer to the second catalyst layer is from 1:4 to 4:1.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the examples and detailed description.

### BRIEF DESCRIPTION OF THE FIGURE

The following figure forms part of the present specification and is included to further demonstrate certain aspects of the present disclosure. The disclosure may be better understood by reference to the figure in combination with the detailed description and examples.

FIG. 1 presents a schematic block diagram of a representative reactor system for sequentially and independently producing a mercaptan and an asymmetrical sulfide.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the systems, processes, or methods described herein are contemplated with or without explicit description of the particular combination. Additionally, unless explicitly recited otherwise, any aspect or feature disclosed herein can be combined to describe inventive systems, processes, or methods consistent with the present disclosure.

In this disclosure, while systems and processes are described in terms of “comprising” various components or steps, the systems and processes also can “consist essentially of” or “consist of” the various components or steps, unless stated otherwise.

The terms “a,” “an,” and “the” are intended to include plural alternatives, e.g., at least one. For instance, the disclosure of “a catalyst” is meant to encompass one catalyst, or mixtures or combinations of more than one catalyst, unless otherwise specified.

Several types of ranges are disclosed herein. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of first and second symmetrical sulfides in the second feed mixture can fall within a range from 10:1 to 1:10. By a disclosure that the molar ratio of first and second symmetrical sulfides in the second feed mixture can range from 10:1 to 1:10, the intent is to recite that the molar ratio can be any ratio within the range and, for example, can include any range or combination of ranges from 10:1 to 1:10, such as from 5:1 to 1:5, from 4:1 to 1:4, from 3:1 to 1:3, from 2:1 to 1:2, or from 1.5:1 to 1:1.5, and so forth. Likewise, all other ranges disclosed herein should be interpreted in a manner similar to this example.

In general, an amount, size, formulation, parameter, range, or other quantity or characteristic is “about” or “approximate,” whether or not it is expressly stated to be such. Whether or not modified by the term “about” or “approximately,” the claims include equivalents to the quantities or characteristics.

Process steps comprising flowing a feed mixture through the catalyst bed can be described in terms of a weight hourly space velocity (WHSV), or the ratio of the weight of a component in the feed mixture that comes in contact with a given weight of catalyst per unit time (which may be expressed in units of 1b/1b/hr or, alternatively, in units of g/g/hr). Herein, WHSV refers to total feed mixture flow rate through the catalyst, which in certain aspects may be represented as the total feed mixture flowing through the total amount of the supported catalyst in the fixed bed reactor, or a defined weight of feed mixture flowing through a unit weight of catalyst over a defined period of time, e.g., in units of 1b/1b/hr.

All disclosed product yields are based on the limiting reactant in the respective reaction, unless explicitly stated otherwise. For example, the limiting reactant in a process for synthesizing an asymmetrical sulfide can be diethyl sulfide and, therefore, the conversions and yields are based on the initial quantity of diethyl sulfide.

Although any methods, devices, and materials similar or equivalent to those described herein can be used in the practice or testing of the disclosure, the typical methods, devices, and materials are herein described.

All publications and patents mentioned herein are incorporated by reference in their entirety for the purpose of describing and disclosing, for example, the constructs and methodologies that are described in the publications and patents, which might be used in connection with the present disclosure.

### DETAILED DESCRIPTION OF THE INVENTION

Disclosed herein are processes for producing mercaptans and asymmetrical sulfides in a fixed bed reactor utilizing a stacked bed of a first catalyst layer comprising a supported cobalt-molybdenum (CoMo) catalyst and a second catalyst layer comprising a supported nickel-molybdenum (NiMo) catalyst. Reactors comprising the CoMo/NiMo stacked catalyst layers are capable of efficiently producing mercaptans and asymmetrical sulfides, independently, within a single reactor without needing to change the catalyst bed and/or needing multiple reactors.

### CATALYSTS

Supported CoMo and NiMo catalysts as generally described herein will be understood to refer to cobalt-molybdenum catalysts and nickel-molybdenum catalysts supported on any suitable solid oxide or like material as disclosed, for instance, in U.S. Pat. Nos. 4,277,623 and 10,927,074; and U.S. Pat. App. Pub. No. 2022/0106266. As an example, supported CoMo catalysts can contain about 2-4 wt. % cobalt and about 8-16 wt. % molybdenum supported on alumina, although the respective amounts of Co and Mo are not limited thereto. Similarly, supported NiMo catalysts can contain about 2-4 wt. % nickel and about 11-17 wt. % molybdenum supported on alumina. CoMo and NiMo catalysts are not limited to any particular ratio of cobalt or nickel to molybdenum components, or by the type of the support. The absolute and relative amounts of the supported CoMo and NiMo catalysts in each of the first and second catalyst layers also may be any suitable amount. In certain aspects, the ratio of the amount of catalyst in the first layer to the amount of catalyst in the second layer can be in a range from 4:1 to 1:4, from 3:1 to 1:3, from 2:1 to 1:2, from 4:1 to 1:1, or from 1:1 to 1:4. In this manner, while the amount of catalyst in the first and second catalyst layers are not necessarily equal, the desired synergistic effect observed by flowing the feed stream first through the first catalyst layer comprising a supported CoMo catalyst prior to flowing the feed stream through the second catalyst layer comprising a supported NiMo catalyst may still be achieved. As will be understood by results and description throughout this disclosure, these benefits become relatively more significant with increasing scale or production (and higher WHSV).

### SYNTHESIZING MERCAPTANS AND ASYMMETRICAL SULFIDES

Processes for producing mercaptans disclosed herein can comprise flowing a first feed mixture through a fixed catalyst bed comprising a two-layer CoMo/NiMo catalyst as described herein. In certain aspects, the first feed stream can comprise H₂Sand an olefin in amounts and relative amounts as disclosed in U.S. Pat. App. Pub. No. 2022/0106266. Non-limiting examples of olefins that can be a reactant in the first feed mixture include ethylene, propylene, a butene, a pentene, a hexene, a heptene, an octene, a decene, a dodecene, a tetradecene, a hexadecene, an octadecene, cyclopentene, cyclohexene, and the like, as well as combinations thereof. Illustrative and non-limiting examples of mercaptans produced by the processes disclosed herein can include methyl mercaptan, ethyl mercaptan, isopropyl mercaptan, sec-butyl mercaptan, and the like, as well as combinations thereof.

Processes for producing an asymmetrical sulfide by transsulfidation of first and second symmetrical sulfides can comprise flowing a second feed stream comprising the first symmetrical sulfide and the second symmetrical sulfide through the fixed catalyst bed—the same utilized for the production of mercaptans described directly above. In this manner, transsulfidation can produce an asymmetrical sulfide containing one of each of the sulfide groups present in the first and second symmetrical sulfide reactants. In certain aspects, the first and second symmetrical sulfides independently can comprise dimethyl sulfide, diethyl sulfide, dibutyl sulfide, dioctyl sulfide, or any combination thereof (with the first symmetrical sulfide being different from the second symmetrical sulfide). For instance, the first symmetrical sulfide can comprise dimethyl sulfide and the second symmetrical sulfide can comprise diethyl sulfide. Thus, the asymmetrical sulfide produced by the transsulfidation process can comprise methyl ethyl sulfide.

The molar ratio of first and second symmetrical sulfides in the second feed stream is not particularly limited, and generally can fall within a range from 10:1 to 1:10. Typical ranges for the molar ratio of symmetrical sulfides in the feed stream can include, but are not limited to, from 5:1 to 1:5, from 4:1 to 1:4, from 3:1 to 1:3, from 2:1 to 1:2, or from 1.5:1 to 1:1.5. Generally, the molar ratio may be selected based on the cost or availability, or the difficulty to isolate, one of the symmetrical sulfides in the second feed mixture, such that the component is mostly consumed during the process. For instance, dimethyl sulfide may be available in excess in production plants, and so can be provided in high molar ratio (e.g., about 4:1) with respect to a second symmetrical sulfide in the second feed stream, such as diethyl sulfide. In such circumstances, the molar ratio of the first and second symmetrical sulfides can range from 1:1 to 10:1, from 2:1 to 10:1, from 2:1 to 5:1, or from 3:1 to 5:1.

Surprisingly, it was found that low amounts of carbon disulfide can improve the catalytic activity of CoMo/NiMo catalysts disclosed herein, as evidenced by improved conversion and yield of processes performed in the presence of carbon disulfide. Thus, in certain aspects, the first and/or second feed stream also can comprise an amount of carbon disulfide in a range from 500 parts per million by weight (ppmw) to 50,000 ppmw, from 1,000 ppmw to 10,000 ppmw, from 3,000 ppmw to 7,500 ppmw, or from 3,500 ppm to 6,500 ppmw. Processes disclosed herein can further comprise adding an amount of carbon disulfide (CS₂) to the first feed stream, the second feed stream, or both, in any amount necessary to achieve the carbon disulfide concentrations disclosed above. For example, carbon disulfide can be added to the first and/or second feed stream in an amount in a range from 500 ppmw to 25,000 ppmw, from 1000 ppmw to 10,000 ppmw, or from 1,500 ppmw to 4,500 ppmw.

The operating conditions of the fixed bed reactor containing the CoMo/NiMo stacked catalyst bed for the production of the mercaptan can be different from the operating conditions of the fixed bed reactor containing the CoMo/NiMo stacked catalyst bed for the production of the asymmetrical sulfide. Representative and non-limiting ranges for the temperature at which the first feed mixture is flowed through the fixed bed reactor and contacted with the CoMo/NiMo stacked catalyst bed (or the temperature at which the mercaptan is formed in step (a) can include from 100° C. to 300° C., from 125° C. to 275° C., from 175° C. to 275° C., from 175° C. to 250° C., from 200° C. to 300° C., from 200° C. to 275° C., or from 200° C. to 250° C. These temperature ranges also are meant to encompass circumstances where the first feed mixture is contacted with the CoMo/NiMo stacked catalyst bed (or where the mercaptan compound is formed) at a series of different temperatures, instead of at a single fixed temperature, falling within the respective temperature ranges, wherein at least one temperature is within the recited ranges.

Temperatures at which the second feed mixture is flowed through the fixed bed reactor and contacted with the CoMo/NiMo stacked catalyst bed (or the temperature at which the asymmetrical sulfide is formed in step (b) can include the same ranges as those disclosed above for step (a) and the production of the mercaptan, or in certain aspects, at a higher temperature. For instance, the production of mercaptan can be performed at a relatively low temperature to minimize the effect of the supported CoMo catalyst in the catalyst bed, thereby allowing the first feed mixture to proceed through the catalyst bed without as much interaction with the CoMo catalyst. In such aspects, the catalyst bed may operate as if the catalyst bed contained only NiMo. However, significant benefits to transsulfidation reactions may be realized where the CoMo catalyst reacts with the second feed mixture. In such aspects, the second feed mixture is flowed through the catalyst bed at higher temperatures than the first reaction mixture. For instance, the second feed mixture can be flowed through the fixed bed reactor and contacted with the CoMo/NiMo stacked catalyst bed (or the asymmetrical sulfide can be formed in step (b) at a temperature from 200° C. to 500° C., such as from 225° C. to 450° C., from 250° C. to 350° C., from 250° C. to 300° C., from 270° C. to 300° C., or from 280° C. to 290° C.

The interaction between the first and second feed mixture and the catalyst bed can be proportional to any of the flow rate(s) of the respective feed stream, the amount of catalyst in the fixed bed reactor, and the concentration of the reactants in the respective feed mixtures. Process steps of flowing the respective feed mixture through the catalyst bed therefore can be described in terms of a weight hourly space velocity (WHSV) as defined herein. The WHSV at which the first or second feed mixtures are flowed through the reactor, producing a mercaptan or an asymmetrical sulfide, respectively, can have a minimum value of 0.01, 0.1, 0.2, 0.5, 1, or 2; or alternatively, a maximum value of 10, 8, 7, 5, 4, or 2. Generally, the WHSV can be in a range from any minimum WHSV disclosed herein to any maximum WHSV disclosed herein. In a non-limiting aspect, the WHSV in step (a) and/or step (b) can be in a range from 0.1 to 5; alternatively, from 1 to 5; or alternatively, from 2 to 4. Other WHSV ranges are readily apparent from this disclosure. While not being limited thereto, step (a) and step (b) can be performed, independently, at a reaction pressure in a range from 25 pounds per square inch gauge (psig) to 1000 psig (172 kPag to 6890 kPag). Other representative and non-limiting ranges for the reaction pressure can include from 25 to 500 psig (172 kiloPascal gauge (kPag) to 3447 kPag), from 50 psig to 800 psig (344 kPag to 5515 kPag), from 50 psig to 450 psig (344 kPag to 3103 kPag), from 200 psig to 450 psig (1379 kPag to 3103 kPag), from 50 psig to 250 psig (344 kPag to 1722 kPag), or from 100 psig to 200 psig (689 kPag to 1379 kPag).

Advantageously, the reaction conditions for step (a) and step (b) are independent, and can be tailored to the desired mercaptan product and the desired asymmetrical sulfide product. In certain aspects, step (a) can be performed at a temperature in a range from 150° C. to 250° C., a pressure in a range from 350 psig to 550 psig, and a WHSV from 0.2 to 1. In certain aspects, step (b) can be performed at a temperature in a range from 270° C. to 300° C., a pressure in a range from 100 to 200 psig, and a WHSV in a range from 2 to 4.

Generally, the processes described herein result in an unexpectedly high conversion of the limiting reactants and/or yield to the asymmetrical sulfide or mercaptan products, particularly where WHSV is reduced for a greater period of contact of the reactants with the catalyst bed. In one aspect, the minimum conversion (or yield) can be at least 50%, at least 60%, at least 70%, at least 80%, at least 90%, or at least 95%. Additionally, the maximum conversion (or yield) can be 97%, 98%, 99%, or 99.5%, and can approach 100% conversion of the limiting reactant (or yield of the asymmetrical sulfide and/or the mercaptan). Generally, the conversion (or yield) can be in a range from any minimum conversion (or yield) disclosed herein to any maximum conversion (or yield) disclosed herein. Non-limiting ranges of conversion (or yield) can include from 50% to 99.5%, from 80% to 99%, from 90% to 98%, or from 95% to 100%.

When controlling for a higher WHSV (e.g., about 3 or more), conversion and yield remain exceptional. Non-limiting ranges of conversion of the asymmetrical sulfide (e.g., diethyl sulfide) at a WSHV of 3 can include from 65% to 95%, from 75% to 80%, from 80% to 90%, or from 82% to 88%. These conversions are contemplated at any suitable reaction conditions disclosed above, for instance at a reaction temperature of 285° C. and a pressure of 150 psig, though not limited thereto. For conversion, the percentages are the amount of the limiting reactant converted based on the initial amount of the limiting reactant. The yield values are mole percentages, and are based on the moles of the asymmetrical sulfide produced (e.g., moles of methyl ethyl sulfide in examples disclosed herein) to moles of the limiting reactant (e.g., diethyl sulfide).

Without being bound by theory, it is believed that utilizing relatively mild reaction conditions during producing the mercaptan can cause the stacked CoMo/NiMo stacked bed catalyst to approach the performance of a 100% NiMo catalyst, as disclosed within U.S. Pat. App. Pub. No. 2022/0106266. As a non-limiting example, processes to produce the mercaptan can be conducted at a reactor temperature of less than 235° C. to prevent significant interaction between the supported CoMo catalyst layer. In this manner, the production of mercaptan and asymmetrical sulfides may be unexpectedly well-suited to be performed within a single reactor system comprising a CoMo/NiMo layered catalyst bed. Surprisingly, the advantages to production of asymmetrical sulfides using the CoMo/NiMo stacked bed catalyst disclosed herein do not require a sacrifice of yields and conversion during production of mercaptans in the subsequent processes, relative to processes to produce mercaptans using 100% NiMo catalyst beds.

Thus, mercaptan syntheses disclosed herein can operate under reactor conditions where the CoMo catalyst bed may have little to no activity on the first reagent stream comprising H₂S and an olefin. In certain aspects, processes to produce a mercaptan can comprise flowing the first reagent stream through the reactor at a temperature in a range from 100° C. to 300° C., from 175° C. to 275° C., or from 150° C. to 250° C. In other aspects, flowing the first reagent stream through the reactor can be conducted at a reactor pressure in a range from 50 psig to 1000 psig, from 100 psig to 800 psig, from 300 psig to 600 psig, or from 200 psig to 400 psig. In certain aspects, the molar ratio of H₂S:olefin can be in a range from 3:1 to 30:1, from 3:1 to 10:1, from 4:1 to 30:1, from 5:1 to 20:1, or from 10:1 to 15:1. In certain aspects, flowing the first reagent stream comprising a mercaptan compound through the reactor can comprise a WHSV in a range from 0.01 to 3, from 0.05 to 1.5, from 0.2 to 1, from 0.01 to 10, from 1 to 10, or from 2 to 4.

In addition to the above characterizations of yield and conversion, the production of asymmetrical sulfides and mercaptans as disclosed herein can be characterized relative to a given set of reaction conditions and other catalyst systems. For example, the conversion of a symmetrical sulfide (or yield of a resulting asymmetrical sulfide) using a stacked bed catalyst comprising a first catalyst layer comprising a supported CoMo catalyst and a second catalyst layer comprising a supported NiMo catalyst can be greater than that of an otherwise identical process comprising a different catalyst composition or arrangement. In certain aspects, the conversion or yield can be greater than (e.g., at least 0.5% greater than, at least 1% greater than, at least 2% greater than, at least 3% greater than, or at least 5% greater than) that observed using (1) a single layer supported NiMo catalyst, or (2) a single layer supported CoMo catalyst, or (3) a mixed catalyst bed comprising a mixture of a supported NiMo catalyst and a supported CoMo catalyst, or (4) a stacked bed catalyst where the order of the NiMo catalyst and the CoMo catalysts is reversed (i.e., the first catalyst layer comprises the supported NiMo catalyst and the second catalyst layer comprises the supported CoMo catalyst), or any combination thereof.

Surprisingly, while processes utilizing the CoMo/NiMo stacked bed catalyst demonstrated a synergistic improvement in the conversion of the limiting symmetrical sulfide reactant and yield of the resulting asymmetrical sulfide, it was also observed that processes disclosed herein did not produce an appreciable increase the amount of mercaptan by-products present in the second reaction mixture. Moreover, the amount of H₂S is effectively unchanged relative to processes with lower conversions of the limiting symmetrical sulfide reactant. In certain processes contemplated herein, an amount of ethylene, H₂S, methyl mercaptan or methanethiol (MeSH), ethyl mercaptan or ethanethiol (EtSH), or any combination thereof, can be within 1% of, within 0.5% of, within 0.3% of, within 0.2% of, within 0.1% of, equal to, or less than that of processes to produce an asymmetrical sulfide that utilize (1) a single layer supported NiMo catalyst, or (2) a single layer supported CoMo catalyst, or (3) a mixed catalyst bed comprising a mixture of a supported NiMo catalyst and a supported CoMo catalyst, or (4) a stacked bed catalyst where the order of the NiMo catalyst and the CoMo catalysts is reversed (i.e., the first catalyst layer comprises the supported NiMo catalyst and the second catalyst layer comprises the supported CoMo catalyst), or any combination thereof.

In many instances, it can be desirable to isolate the asymmetrical sulfide and/or the mercaptan from the respective reaction mixture for sale or for use in further industrial processes. Accordingly, in certain aspects, the process for producing an asymmetrical sulfide and a mercaptan can further comprise a step of isolating the asymmetrical sulfide (or isolating the mercaptan compound) from the respective reaction mixture to form a product stream containing the asymmetrical sulfide (or a product stream containing the mercaptan) by any suitable technique. Suitable techniques can include, but are not limited to, extraction, filtration, evaporation, or distillation, as well as combinations of two or more of these techniques. In particular aspects of this disclosure, the isolating step utilizes distillation at any suitable pressure (where one, or more than one, distillation column can be used). Advantageously, the low levels of mercaptans in the second reaction mixture make isolating, for instance, an asymmetrical sulfide such as methyl ethyl sulfide via distillation a relatively straightforward process. After the isolating step, the asymmetrical sulfide (or the mercaptan) can have a purity of at least 80 wt. %, at least 85 wt. %, at least 90 wt. %, at least 95 wt. %, or at least 98 wt. %, in the product stream.

### REACTOR SYSTEMS

A reactor system consistent with this disclosure can comprise (i) a fixed bed reactor comprising a reactor inlet for a feed stream, a first catalyst layer comprising a supported CoMo catalyst, a second catalyst layer comprising a supported NiMo catalyst, and a reactor outlet for a reaction mixture, (ii) a first feed source comprising a H₂S storage vessel and an olefin storage vessel, and (iii) a second feed source comprising a first symmetrical sulfide storage vessel and a second symmetrical sulfide storage vessel. In this reactor system, a flow path for the feed stream begins at the reactor inlet, through the first catalyst layer and then the second catalyst layer, and to the reactor outlet, and a weight ratio of the first catalyst layer to the second catalyst layer ranges from 1:4 to 4:1.

Catalysts suitable for the reactor systems disclosed herein can be any as described above and suitable for producing mercaptans and asymmetrical sulfides, and for performing downstream processing. For instance, in certain aspects the supported CoMo catalyst and the supported NiMo catalyst each can independently be supported on silica, alumina, silica-alumina, aluminum phosphate, zinc aluminate, zirconia, thoria, and the like. In certain aspects, either or both of the supported CoMo catalyst and the supported NiMo catalyst can comprise an alumina support.

Surprisingly, reactor systems disclosed herein are able to accommodate the production of mercaptans and asymmetrical sulfides within a single reactor system and without the need for separate equipment or interchanging catalysts and flow paths through the catalyst bed. Integrated catalyst systems have been previously described to produce both mercaptan and asymmetric sulfide via separate reactors as part of a carefully orchestrated system of feed streams, products, and recycled byproducts. Previously disclosed integrated systems are therefore limited by their reliance on independent reactors for the synthesis of mercaptans and asymmetric sulfides.

In contrast, reactor systems disclosed herein can be configured for the production of mercaptan and asymmetrical sulfide within a single reactor, including arrangements encompassing two independent reagent and product streams within the single reactor. In certain aspects, reactor systems can comprise a feed selector valve positioned in the feed stream between the fixed bed reactor (e.g., the reactor inlet of a fixed bed reactor) and the first and second feed sources. The feed selector valve can be in fluid communication with each of the first and second feed sources and the reactor inlet, and therefore may adopt a first position where the first feed stream is delivered from the first feed source to the reactor inlet. The feed selector valve also can adopt a second position where the second feed stream is delivered from the second feed source to the reactor inlet. In this manner each of the first and second feed streams may be delivered to the fixed bed reactor for sequential and independent processes to produce the mercaptan and asymmetrical sulfides as disclosed herein.

Similarly, reaction systems disclosed herein can comprise a product selector valve positioned between the reactor outlet and the first and second product streams of the reactor system. As for the feed selector valve, the product selector valve can adopt a first position delivering a reaction mixture from the reactor outlet to either of the first or second product stream lines. In this manner, a single catalyst bed (e.g., a catalyst bed comprising supported CoMo/NiMo) can receive feed streams relevant to both mercaptan synthesis and asymmetrical sulfide synthesis, and deliver reaction mixtures to their respective downstream processes without need for interchanging components or catalysts within the reactor system.

Reactor systems disclosed herein can comprise additional components arranged as necessary to maintain reaction conditions as disclosed above. For instance, with respect to the temperature within the reactor and catalyst bed, the fixed bed reactor can comprise a heating jacket surrounding the catalyst bed to control the temperature within the reactor and/or to maintain the temperature within an intended range for producing the mercaptan or asymmetrical sulfide. Additionally, or alternatively, the feed source line can comprise a preheater to raise the temperature of reagent mixtures before entering the reactor and to ensure that the temperature within the reactor is maintained throughout the reaction, even as the feed stream enters the reactor. Reactor components to modulate and maintain reaction pressures and additional reaction parameters (e.g., feed stream flow rate, reagent concentration) are also contemplated herein and would be understood by those of skill in the art.

Controllers and analytical modules to monitor reactor conditions, feed streams, product streams, and the like are also contemplated within reactor systems disclosed herein, as will be understood by those of skill in the art. The first and second feed sources are not limited to any particular source, and may be any that provide the feed stream or its components in a form that is suitable for the reaction.

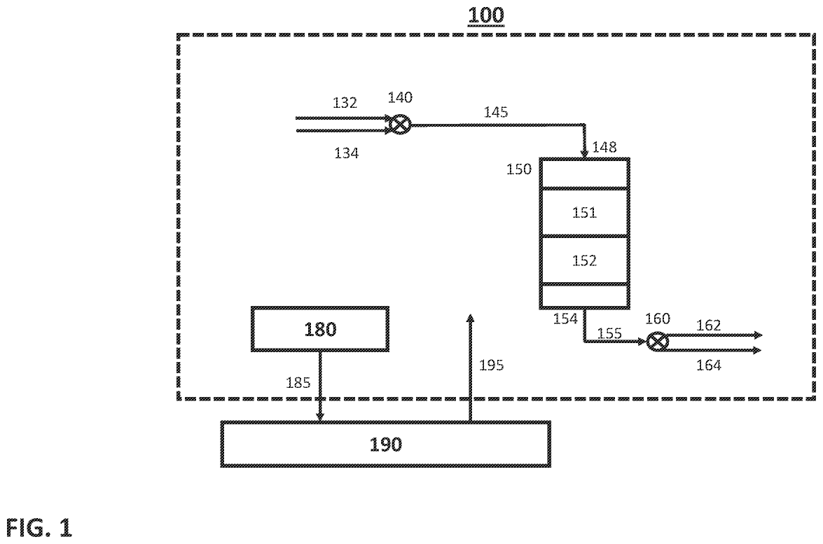

A representative fixed bed reactor system 100 consistent with aspects of this disclosure is illustrated in FIG. 1. As shown, reactor system 100 includes fixed bed reactor 150 with feed stream inlet 148 leading into the reactor and downward toward reaction mixture outlet 154. As will be understood by those of skill in the art, fixed bed reactor 150 may be fitted with jacket heaters to maintain a suitable reaction temperature within the reactor. Fixed bed reactor 150 comprises the two-layer catalyst bed as described above, with first catalyst layer 151 comprising a supported CoMo catalyst positioned to first contact the downward feed stream flow from inlet 148 to outlet 154 before contacting a second catalyst layer 152 comprising a supported NiMo catalyst. First reactant line 132 and second reactant line 134 enter a feed selector valve 140, which further directs the selected feed stream from either the first reactant line 132 or the second reactant line 134 into reactor feed 145 and then into reactor 150 via inlet 148. Similarly, reaction product 155 delivers the product mixture exiting the reactor 150 via reactor outlet 154 to product selector valve 160, which further directs the product stream to the respective first product line 162 or second product line 164.

If selected by feed selector valve 140, first reactant line 132, which is connected to a H₂₅ storage vessel and an olefin storage vessel (not shown), delivers a H₂S-olefin feed stream to the reactor 150, thereby producing a mercaptan product, which is conveyed to first product line 162 thru product selector value 160. Similarly, if selected by feed selector valve 140, second reactant line 134, which is connected to a first symmetrical sulfide storage vessel and a second symmetrical sulfide storage vessel (not shown), delivers a mixed sulfide feed stream to the reactor 150, thereby producing an asymmetrical sulfide product, which is conveyed to second product line 164 thru product selector value 160. Storage vessels referred to herein can comprise one or more physical storage containers (e.g., a tank) capable of long term storage and periodic replenishment. In other aspects, for instance in integrated reactor systems, the storage vessels contemplated herein can comprise a crude or purified product stream of an integrated reactor system.

Reactor system 100 also can include sensors configured to monitor reaction parameters (e.g., temperature, pressure, flow rate) and status of components within the reactor system 100. Analytical module 180 is provided as a means for receiving system information (e.g., reaction parameters) and transmitting data 185, shown in connection with controller 190, which then controls or adjusts via control output 195 (e.g., increases or decreases reaction temperature, increases or decreases feed stream flow rate into the reactor) any aspect of the reactor system 100 based on data 185 from analytical module 180.

### EXAMPLES

The disclosure is further illustrated by the following examples, which are not to be construed in any way as imposing limitations to the scope of this disclosure. Various other aspects, modifications, and equivalents thereof, which after reading the description herein, can suggest themselves to one of ordinary skill in the art without departing from the spirit of the present disclosure or the scope of the appended claims. These examples demonstrate that reaction conditions favorable for mercaptan syntheses also can produce favorable results in transsulfidation reactions utilizing a two-layer catalyst comprising a first (top) layer comprising a supported CoMo catalyst and a second (bottom) layer comprising a supported NiMo catalyst.

In each of Examples 1-16, transsulfidation reactions were conducted using a feed stream prepared by blending dimethyl sulfide (DMS) and diethyl sulfide (DES) at a molar ratio of 4:1. The DMS and DES blend was fed into the top of a fixed bed reactor containing the appropriate catalyst. Carbon disulfide was also present in the feed stream as residual from the production of dimethylsulfide, in an amount of approximately 3000 ppmw (0.3 wt. %). For Examples 1-12, a two-layer catalyst bed was employed, (i) supported CoMo on alumina and (ii) supported NiMo on alumina, in a 1:1 weight ratio. Examples 13-16 examined alternative catalyst arrangements as discussed in detail below.

Results of the experiments performed in Examples 1-16 are provided in Tables I-III, and were determined by gas chromatography (GC) performed on an Agilent 7890A GC system (Agilent Technologies, Wilmington, DE, USA), using an Agilent HP-5 GC column (dimethylpolysiloxane, capillary 30 m ×0.32 μm ×0.25 nominal), programmed with a 35° C. temperature hold for 5 min, followed by ramping at a rate of 5° C./min from 35° C. to 70° C., followed by ramping at 15° C./min to 260° C., then holding at 260° C. for 10 min. Standards for dimethyl sulfide (DMS), diethyl sulfide (DES), and methyl ethyl sulfide (IVIES) were used to identify the respective reactants and products. WHSV was calculated as described herein, on the basis of the total weight of DES contacted with the catalyst per unit time.

Unexpectedly, stacked bed catalysts comprising a CoMo catalyst as a first layer and a NiMo catalyst as a second layer converted high levels of diethyl sulfide (e.g., greater than 80-85 mol %), even under conditions favorable to mercaptan synthesis, and produced only a minimal amount of light by-products and unreacted reactants in the product mixture. Examples 1-16 demonstrate transsulfidation reactions converted DMS and DES primarily to methyl ethyl sulfide (MES).

### Examples 1-6

Evaluation of favorable asymmetrical sulfide reaction conditions to complement mercaptan synthesis protocol.

In Examples 1-6, reaction conditions for the transsulfidation were varied according to temperature, pressure, and reactant flow rate through the reactor (weight hourly space velocity (WHSV)). Each of Examples 1-6 was conducted using a stacked bed catalyst comprising a first (top) layer of a CoMo catalyst and a second (bottom) layer of a NiMo catalyst. Results are shown in Table I.

Examples 1-3 examined the effect of temperature on the transsulfidation reaction, holding pressure and flow rate/WSHV constant. As may be expected, the conversion of the reactants generally increased as temperature increased from 270° C. to 300° C., and surprisingly the amount of by-products increased significantly at 300° C., such that the overall yield of methyl ethyl sulfide decreased above 285° C. A temperature of 270° C. also saw a marked decrease in the amount of unwanted lights (e.g., H₂S, MeSH, EtSH) in the product mixture, however, this decrease was accompanied by a decrease in the conversion of diethyl sulfide and accordingly the yield of methyl ethyl sulfide.

Example 4 examined the effect of increasing the flow rate of the reactant mixture through the catalyst bed. As expected, the conversion of diethyl sulfide dropped significantly compared to the results exemplified in Example 2 due to the reduced contact time with the catalyst. The amount of lights produced as unwanted by-products was not drastically decreased, and EtSH was produced in a higher amount.

Examples 5-6 examined the effect of reducing the pressure within the reactor. While lower pressures would be expected to cause lower yields and conversion of the limiting reactant, conducting transsulfidation reactions at lower pressures may beneficially allow the transsulfidation reactions to be performed on equipment conventionally utilized for mercaptan synthesis. Examples 5-6 were conducted at reduced pressures of 150 psig and 100 psig, respectively. Surprisingly, the conversion of diethyl sulfide remained above 80% despite the reduction of reaction pressure to 150 psig, which represents only 50% of the pressure in Examples 1-4. Similarly, the diethyl sulfide conversion observed in Example 6 (100 psig) was also nearly 80%. Further, the amount of lights produced was reduced relative to higher pressure examples. Thus, the excellent results of transsulfidation reactions under relatively mild conditions shows that the reaction may be conducted in reactor systems typically employed under lower pressures and temperatures, such as equipment dedicated to the production of mercaptan syntheses.

### Examples 7-12

Evaluation of the effect of added CS₂ and recycled lights within the asymmetrical sulfide feed stream.

In Examples 7-12, components of the feed stream were examined for their effect on the conversion and amount of by-products produced. Similar to Examples 1-6, each of Examples 7-12 was conducted using the same stacked bed catalyst comprising a first (top) layer of a CoMo catalyst and a second (bottom) layer of a NiMo catalyst. Results are shown in Table II.

Examples 7-8 examined the effect of carbon disulfide present in the reaction feed. Example 7 represents a feed mixture having a low CS₂ concentration, i.e., below 3000 ppmw. Example 8 provided an additional 1905 ppmw CS₂ to the feed stream of Example 7. As shown in Table II, the conversion of the low-CS₂ reactant feed was significantly lower than for Example 8, where CS₂ was added to the feed stream.

Examples 9-12 examined the effect of lights in the reactant feed stream, which may be present from recycling certain components of the product stream. In closed reactor systems, the unwanted lights produced would need to be separated from the product mixture (e.g., by fractionation) and either disposed of as waste or, preferably, recycled within the process. In Examples 9-11, MeSH was added to the feed stream to an amount of 2.2 wt. %. This MeSH-added feed stream was also examined with low CS₂ levels (no added CS₂, less than 3000 ppmw total CS₂), moderate CS₂ levels (1905 ppmw CS₂ added, from 3000 ppmw to 5000 ppmw total CS₂), and high CS₂ levels (3810 ppmw CS₂ added, from 5000 to 7000 ppmw total CS₂). Example 12 employed a feed stream with 0.7% EtSH, and 0.1 wt. % CS₂.

The amount of CS₂ in the feed stream initially improved the conversion, but the improvement appeared to diminish after reaching an amount of about 5000 ppmw. Specifically, the conversion of diethyl sulfide observed in the moderate CS₂ feed stream of Example 10 was roughly 8% higher than the low CS₂ feed stream (Example 9). Yet, increasing the CS₂ by the same amount in high-CS₂ Example 11 led to a further increase of less than 2%.

Surprisingly, it was also noted that the presence of lights by-products in transsulfidation feed streams of Examples 9-12 was generally well-tolerated, despite being added in amounts that were roughly three to five times the amount observed in the transsulfidation product mixtures examined, such as in Example 8. Accordingly, it was determined that the unwanted lights were produced in amounts that can be recycled into feed streams without significant impact on the conversion or yield of those processes.

### Examples 13-16

Evaluation of asymmetrical sulfide synthesis under alternative arrangements of CoMo and NiMo catalysts.

In Examples 13-16, the composition and arrangement of catalyst within the fixed bed reactor were examined for its impact on the conversion, yield, and production of lights as by-products in the product mixture. Examples 13-16 are summarized in Table III, in comparison to Example 8, which utilized a stacked bed catalyst comprising a first (top) layer of a CoMo catalyst and a second (bottom) layer of a NiMo catalyst.

Example 13 utilized a stacked bed catalyst where the position of the first and second catalyst layers was reversed relative to Example 8, i.e., the stacked bed catalyst consisted of a first (top) layer of a supported NiMo catalyst and a second (bottom) layer of a supported CoMo catalyst. Example 14 utilized a single catalyst layer consisting of a mixture of the supported CoMo catalyst and the supported NiMo catalyst in a 1:1 wt. ratio. Examples 15 and 16 utilized a single catalyst layer with only the supported CoMo catalyst, or only the supported NiMo catalyst, respectively.

As shown in Table III, a reduced conversion was observed in each of the alternative catalyst arrangements of Examples 13-16, relative to Example 8. Surprisingly, the CoMo/NiMo stacked bed catalyst of Example 8 outperformed each of Examples 13-16 with respect to diethyl sulfide conversion and methyl ethyl sulfide yield. Particularly, each of Examples 8, 13-14, and 16, which employed at least some portion of supported NiMo catalyst within the catalyst bed, drastically outperformed the 100% CoMo catalyst utilized in Example 15, suggesting that supported NiMo catalysts broadly may be more effective for transsulfidation than analogous CoMo catalysts. This is further supported by Examples 13-14, in which a slight reduction in conversion and yield relative to the 100% NiMo catalyst of Example 16 was observed, and may be attributed to proportional dilution of efficacy by inclusion of the CoMo catalyst.

Surprisingly, using CoMo as a first layer of the CoMo/NiMo stacked bed catalyst (Example 8) outperformed each of Examples 13-16, including Example 15, in which a 100% NiMo catalyst bed was used. Further, Example 8 showed no increase in the amount of H₂Sproduced during the reaction, and only moderate increases in the amount of MeSH and EtSH relative to Examples 13-16. Without being bound by theory, it is therefore believed that the catalyst bed including a first layer of CoMo provides a synergistic benefit to the transsulfidation reaction when accompanied by the NiMo catalyst as a second layer in the catalyst bed.

TABLE I
Summary of results from Examples 1-6.

| | Temp. | Pressure | | H₂S | MeSH | EtSH | DMS | DES | MES | DES |
|---|---|---|---|---|---|---|---|---|---|---|
| Example | (° C.) | (psig) | WHSV | (wt %) | (wt %) | (wt %) | (wt %) | (wt %) | (wt %) | Conversion (%) |
| 1 (4 runs) | 270 | 300 | 3 | 0.1 | 0.5 | 0.2 | 54.6 | 7.9 | 36.2 | 79.7 |
| 2 (4 runs) | 285 | 300 | 3 | 0.3 | 0.9 | 0.3 | 53.4 | 5.7 | 39.0 | 85.4 |
| 3 (4 runs) | 300 | 300 | 3 | 0.9 | 1.7 | 0.7 | 52.8 | 5.4 | 37.9 | 86.1 |
| 4 (5 runs) | 285 | 300 | 4 | 0.1 | 0.9 | 0.5 | 52.5 | 6.8 | 37.3 | 80.7 |
| 5 (6 runs) | 285 | 150 | 3 | 0.3 | 0.8 | 0.4 | 54.8 | 6.4 | 36.7 | 81.1 |
| 6 (6 runs) | 285 | 100 | 3 | 0.3 | 0.7 | 0.4 | 55.4 | 6.8 | 35.8 | 79.8 |

TABLE II
Summary of results from Examples 7-12.

| | Temp. | Pressure | Added CS₂ | Ethylene | H₂S | MeSH | EtSH | DMS | DES | MES | DES |
|---|---|---|---|---|---|---|---|---|---|---|---|
| Example | (° C.) | (psig) | (ppmw) | (wt %) | (wt %) | (wt %) | (wt %) | (wt %) | (wt %) | (wt %) | Conversion (%) |
| 7 (5 runs) | 285 | 150 | 0 | 0 | 0.5 | 1.1 | 0.5 | 55.9 | 9.2 | 32.1 | 73.5 |
| 8 (8 runs) | 285 | 150 | 1905 | 0 | 0.2 | 1.6 | 0.9 | 53.3 | 5.4 | 36.9 | 85.1 |
| 9 (6 runs) | 285 | 150 | 0 | 0 | 0.3 | 1.6 | 0.8 | 57.5 | 11.4 | 28.1 | 66.2 |
| 10 (10 runs) | 285 | 150 | 1905 | 0 | 0.1 | 1.6 | 0.7 | 59.6 | 7.9 | 29.7 | 73.4 |
| 11 (6 runs) | 285 | 150 | 3810 | 0 | 0.3 | 1.4 | 0.8 | 55.2 | 8.7 | 33.1 | 75.2 |
| 12 (6 runs) | 285 | 150 | 920 | 0.3 | 0.3 | 1.0 | 0.6 | 53.3 | 7.5 | 36.7 | 77.4 |

TABLE III
Summary of results from Examples 13-16, and comparison to Example 8.

| | Temp. | Pressure | Added CS₂ | Ethylene | H₂S | MeSH | EtSH | DMS | DES | MES | DES |
|---|---|---|---|---|---|---|---|---|---|---|---|
| Example | (° C.) | (psig) | (ppmw) | (wt %) | (wt %) | (wt %) | (wt %) | (wt %) | (wt %) | (wt %) | Conversion (%) |
| 13 (8 runs) | 285 | 150 | 1905 | 0 | 0.2 | 0.8 | 0.4 | 56.8 | 6.8 | 34.7 | 79.2 |
| 14 (7 runs) | 285 | 150 | 1905 | 0.02 | 0.2 | 1.0 | 0.6 | 55.0 | 7.3 | 35.4 | 78.8 |
| 15 (11 runs) | 285 | 150 | 1905 | 0 | 0.2 | 0.8 | 0.3 | 59.0 | 16.3 | 23.0 | 51.8 |
| 16 (8 runs) | 285 | 150 | 1905 | 0.08 | 0.4 | 1.3 | 0.8 | 54.6 | 5.8 | 36.3 | 83.2 |
| 8 (8 runs) | 285 | 150 | 1905 | 0 | 0.2 | 1.6 | 0.9 | 53.3 | 5.4 | 36.9 | 85.1 |

The disclosure is described above with reference to numerous aspects and specific examples. Many variations will suggest themselves to those skilled in the art in light of the above detailed description. All such obvious variations are within the full intended scope of the appended claims. Other aspects of the disclosure can include, but are not limited to, the following (aspects are described as “comprising” but, alternatively, can “consist essentially of” or “consist of”):

Aspect 1. A process for sequentially producing a mercaptan and an asymmetrical sulfide, the process comprising:
(a) flowing a first feed mixture comprising H₂S and an olefin through a first catalyst layer of a fixed bed reactor comprising (or consisting essentially of, or consisting of) a supported CoMo catalyst, then through a second catalyst layer of the fixed bed reactor comprising (or consisting essentially of, or consisting of) a supported NiMo catalyst to produce a first reaction mixture comprising the mercaptan; and
(b) flowing a second feed mixture comprising a first symmetrical sulfide and a second symmetrical sulfide through the first catalyst layer and then the second catalyst layer in the fixed bed reactor to produce a second reaction mixture comprising the asymmetrical sulfide.

Aspect 2. The process of aspect 1, wherein the first feed mixture is flowed through the fixed bed reactor at any temperature from 150° C. to 250° C. and any pressure from 25 psig to 450 psig.

Aspect 3. The process of aspect 1 or 2, wherein the olefin comprises ethylene, propylene, 1-butene, or a combination thereof.

Aspect 4. The process of any one of aspects 1-3, wherein the mercaptan comprises isopropyl mercaptan, sec-butyl mercaptan, or a combination thereof.

Aspect 5. The process of any one of aspects 1-4, wherein the first feed mixture is flowed through the fixed bed reactor at a WHSV in a range from 0.01 to 10 (or from 0.1 to 2). Aspect 6. The process of any one of aspects 1-5, wherein the second feed mixture is flowed through the fixed bed reactor at any temperature from 250° C. to 350° C. (or from 270° C. to 300° C.); any pressure from 25 psig to 400 psig (or from 50 psig to 250 psig); and any WHSV from 0.1 to 10 with respect to the limiting reactant (e.g., diethyl sulfide).

Aspect 7. The process of any one of aspects 1-6, wherein the first symmetrical sulfide and the second symmetrical sulfide independently comprise dimethyl sulfide, diethyl sulfide, dipropyl sulfide, dibutyl sulfide, dioctyl sulfide, or any combination thereof.

Aspect 8. The process of any one of aspects 1-7, wherein the first symmetrical sulfide comprises dimethyl sulfide, the second symmetrical sulfide comprises diethyl sulfide, and the asymmetrical sulfide comprises methyl ethyl sulfide.

Aspect 9. The process of aspect 8, wherein a molar ratio of dimethyl sulfide to diethyl sulfide in the second feed mixture is from 1:1 to 10:1, or from 2:1 to 5:1.

Aspect 10. The process of any one of aspects 1-9, wherein the second feed mixture further comprises from 2,500 ppmw to 10,000 ppmw (or 3,500 ppmw to 6,500 ppmw) carbon disulfide.

Aspect 11. The process of any one of aspects 1-10, wherein the second feed mixture is flowed through the fixed bed reactor at any WHSV from 2 to 4.

Aspect 12. The process of any one of aspects 8-11, wherein a conversion of diethyl sulfide is from 60 mol % to 90 mol %.

Aspect 13. The process of any one of aspects 8-12, wherein a yield of methyl ethyl sulfide in the second mixture is from 38 to 50 wt. %, based on the amount of the diethyl sulfide.

Aspect 14. The process of any one of aspects 1-13, wherein an amount of lights (e.g., H₂S, MeSH, EtSH) in the second reaction mixture is less than or equal to 5 wt. %, or less than or equal to 2 wt. %.

Aspect 15. The process of any one of aspects 1-14, wherein a yield of the asymmetrical sulfide (or conversion of a limiting sulfide reactant) is greater than that of an otherwise identical process, wherein the first catalyst layer is the supported NiMo catalyst and the second catalyst layer is the supported CoMo catalyst, under the same reaction conditions.

Aspect 16. The process of any one of aspects 1-15, wherein a yield of the asymmetrical sulfide (or conversion of a limiting sulfide reactant) is greater than that for an otherwise identical process, wherein the fixed bed reactor comprises a mixed bed of the supported NiMo catalyst and the supported CoMo catalyst, under the same reaction conditions.

Aspect 17. The process of any one of aspects 1-16, wherein a yield of the asymmetrical sulfide (or conversion of a limiting sulfide reactant) is greater than that for an otherwise identical process, wherein the fixed bed reactor comprises a single catalyst layer consisting of the supported NiMo catalyst or the supported CoMo catalyst, under the same reaction conditions.

Aspect 18. The process of any one of aspects 15-17, wherein the second reaction mixture comprises an amount of residual ethylene, methyl mercaptan, ethyl mercaptan, or any combination thereof that is less than that of the otherwise identical process.

Aspect 19. The process of any one of aspects 1-18, wherein the supported NiMo catalyst comprises an alumina support, the supported CoMo catalyst comprises an alumina support, or both.

Aspect 20. The process of any one of aspects 1-19, wherein a weight ratio of the first catalyst layer of the supported CoMo catalyst to the second catalyst layer of supported NiMo catalyst is from 1:4 to 4:1, or from 1:1.5 to 1.5:1.

Aspect 21. A reactor system comprising (i) a fixed bed reactor comprising a reactor inlet for a feed stream, a first catalyst layer comprising a supported CoMo catalyst, a second catalyst layer comprising a supported NiMo catalyst, and a reactor outlet for a reaction mixture, wherein a flow path for the feed stream begins at the reactor inlet, through the first catalyst layer and then the second catalyst layer, and to the reactor outlet, and a weight ratio of the first catalyst layer to the second catalyst layer is from 1:4 to 4:1, (ii) a first feed source comprising a H₂S storage vessel and an olefin storage vessel, and (iii) a second feed source comprising a first symmetrical sulfide storage vessel and a second symmetrical sulfide storage vessel.

Aspect 22. The system of aspect 21, wherein the supported CoMo catalyst is supported on alumina, the supported NiMo catalyst is supported on alumina, or both.

Aspect 23. The system of aspect 21 or 22, wherein the system further comprises a feed selector valve positioned between the fixed bed reactor and the first and second feed sources, the feed selector valve configured to operate between:
a first feed position delivering a first feed stream comprising H₂S and an olefin from the first feed source to the reactor inlet; and
a second feed position delivering a second feed stream comprising a first symmetric sulfide and a second symmetric sulfide from the second feed source to the reactor inlet.

Aspect 24. The system of any one of aspects 21-23, wherein the system further comprises a product selector valve positioned between the fixed bed reactor and first and second product lines, the product selector valve configured to operate between:
a first product position delivering the reaction mixture from the reactor outlet to a mercaptan product stream; and a second product position delivering the reaction mixture from the reactor outlet to an asymmetrical sulfide product stream.

We claim:

1. A process for sequentially producing a mercaptan and an asymmetrical sulfide, the process comprising:
(a) flowing a first feed mixture comprising H₂S and an olefin through a first catalyst layer of a fixed bed reactor comprising a supported CoMo catalyst, then through a second catalyst layer of the fixed bed reactor comprising a supported NiMo catalyst to produce a first reaction mixture comprising the mercaptan; and
(b) flowing a second feed mixture comprising a first symmetrical sulfide and a second symmetrical sulfide through the first catalyst layer and then the second catalyst layer in the fixed bed reactor to produce a second reaction mixture comprising the asymmetrical sulfide.

2. The process of claim 1, wherein the first feed mixture is flowed through the fixed bed reactor at:
a temperature from 150° C. to 250° C.;
a pressure from 25 psig to 450 psig; and
a WHSV from 1 to 10.

3. The process of claim 1, wherein the olefin comprises ethylene, propylene, 1-butene, or a combination thereof.

4. The process of claim 1, wherein the mercaptan comprises isopropyl mercaptan, sec-butyl mercaptan, or a combination thereof.

5. The process of claim 1, wherein the second feed mixture is flowed through the fixed bed reactor at:
a temperature from 250° C. to 350° C.;
a pressure from 25 psig to 400 psig; and
a WHSV from 2 to 4.

6. The process of claim 1, wherein the first symmetrical sulfide and the second symmetrical sulfide independently comprise dimethyl sulfide, diethyl sulfide, dipropyl sulfide, dibutyl sulfide, dioctyl sulfide, or any combination thereof.

7. The process of claim 1, wherein:
the first symmetrical sulfide comprises dimethyl sulfide;
the second symmetrical sulfide comprises diethyl sulfide; and
the asymmetrical sulfide comprises methyl ethyl sulfide.

8. The process of claim 7, wherein a molar ratio of dimethyl sulfide to diethyl sulfide in the second feed mixture is from 2:1 to 5:1.

9. The process of claim 7, wherein the second feed mixture further comprises from 3,500 ppmw to 6,500 ppmw carbon disulfide.

10. The process of claim 7, wherein a conversion of diethyl sulfide is from 60 mol % to 90 mol %.

11. The process of claim 7, wherein a yield of methyl ethyl sulfide in the second reaction mixture is from 38 wt. % to 50 wt. %.

12. The process of claim 1, wherein a conversion of either of the first asymmetrical sulfide or the second asymmetrical sulfide is greater than that of an otherwise identical process, wherein the first catalyst layer is the supported NiMo catalyst and the second catalyst layer is the supported CoMo catalyst, under the same reaction conditions.

13. The process of claim 1, wherein a conversion of either of the first asymmetrical sulfide or the second asymmetrical sulfide is greater than that for an otherwise identical process wherein the fixed bed reactor comprises a mixed bed of the supported NiMo catalyst and the supported CoMo catalyst, under the same reaction conditions.

14. The process of claim 1, wherein a conversion of either of the first asymmetrical sulfide or the second asymmetrical sulfide is greater than that for an otherwise identical process wherein the fixed bed reactor comprises a single catalyst layer consisting of the supported NiMo catalyst or the supported CoMo catalyst, under the same reaction conditions.

15. The process of claim 1, wherein the supported NiMo catalyst comprises an alumina support, the supported CoMo catalyst comprises an alumina support, or both.

16. The process of claim 1, wherein a weight ratio of the first catalyst layer of the supported CoMo catalyst to the second catalyst layer of the supported NiMo catalyst is from 1:4 to 4:1.

17. A reactor system comprising:
(i) a fixed bed reactor comprising:
a reactor inlet for a feed stream;
a first catalyst layer comprising a supported CoMo catalyst;
a second catalyst layer comprising a supported NiMo catalyst; and
a reactor outlet for a reaction mixture;
wherein:
a flow path for the feed stream begins at the reactor inlet, through the first catalyst layer and then the second catalyst layer, and to the reactor outlet; and
a weight ratio of the first catalyst layer to the second catalyst layer is from 1:4 to 4:1;
(ii) a first feed source comprising a H₂S storage vessel and an olefin storage vessel; and
(iii) a second feed source comprising a first symmetrical sulfide storage vessel and a second symmetrical sulfide storage vessel.

18. The system of claim 17, wherein:
the supported CoMo catalyst is supported on alumina;
the supported NiMo catalyst is supported on alumina; or
both.

19. The system of claim 17, wherein the system further comprises a feed selector valve positioned between the fixed bed reactor and the first and second feed sources, the feed selector valve configured to operate between:
a first feed position delivering a first feed stream comprising H₂S and an olefin from the first feed source to the reactor inlet; and
a second feed position delivering a second feed stream comprising a first symmetric sulfide and a second symmetric sulfide from the second feed source to the reactor inlet.

20. The system of claim 17, wherein the system further comprises a product selector valve positioned between the fixed bed reactor and first and second product lines, the product selector valve configured to operate between:
a first product position delivering the reaction mixture from the reactor outlet to a mercaptan product stream; and
a second product position delivering the reaction mixture from the reactor outlet to an asymmetrical sulfide product stream.
